SEQUENCE LISTING (3 sequences)

SEQ ID NO: 1 - DNA, 25 bp, Artificial sequence
  Synthetic oligonucleotide
aaaaaaaaaaatcctttacaatatt

SEQ ID NO: 2 - DNA, 25 bp, Artificial sequence
  Synthetic oligonucleotide
ttttttttttatcctttacaatatt

SEQ ID NO: 3 - DNA, 15 bp, Artificial sequence
  Synthetic oligonucleotide
atcctttacaatatt

(19) United States
(12) Patent Application Publication
(10) Pub. No.: US 2010/0099858 A1
(43) Pub. Date: Apr. 22, 2010

(54) Maximizing Oligonucleotide Loading on Gold Nanoparticle

(76) Inventors: Chad A. Mirkin, Wilmette, IL (US); Abigail K.R. Lytton-Jean, Chicago, IL (US); Sarah J. Hurst, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/441,952

(22) PCT Filed: Sep. 25, 2007
(86) PCT No.: PCT/US07/20644

§ 371 Date: Nov. 9, 2009

Related U.S. Application Data
(60) Provisional application No. 60/847,757, filed on Sep. 28, 2006.

(51) Int. Cl. C07H 21/04

(52) U.S. Cl. 536/23.1; 977/773

(57) ABSTRACT

Increasing the amount of DNA loaded onto gold nanoparticles is disclosed. More particularly, methods of maximizing DNA loading, using salting techniques, sonication, temperature and other such procedures are disclosed.

### CROSS REFERENCE TO RELATED APPLICATIONS

This application claims the benefit of U.S. Provisional Application Ser. No. 60/847,757, filed Sep. 28, 2006, which is incorporated herein in its entirety by reference.

### STATEMENT OF GOVERNMENT INTEREST

This invention was made with U.S. government support under National Science Foundation (NSF-NSEC) Grant No. EEC-011-8025, National Institutes of Health (National Cancer Institute) Grant No. U54 CA 119341-01, and Homeland Security Advanced Research Projects Agency/U.S. Army Medical Research and Material Command Grant No. W81XWH-05-2-0036. The government has certain rights in this invention.

### BACKGROUND

Gold nanoparticles exhibit several interesting physical and chemical properties which have made them an integral part of nanoscience research (Burda, et al. Chem. Rev., 105:1025-1102 (2005)). In addition to their optical properties, gold nanoparticles are important because they can be modified with a wide variety of molecules by taking advantage of well known chemistry involving alkyl thiol adsorption on gold (Love, et al. Chem. Rev., 105:1103-1169 (2005)). In particular, thiol modified oligonucleotides can be loaded onto the surface of gold nanoparticles. The resulting functionalized nanoparticles are widely used as nanoscale building blocks in assembly strategies, as antisense agents in nanotherapeutics for gene regulation, and as probes in many biodiagnostic systems. (Mirkin, et al. Nature, 382:607-609 (1996); Alivisatos, et al. Nature, 382:609-611 (1996); Rosi, et al. Science, 312:1027-1030 (2006); and Rosi, et al. Chem. Rev., 105:1547-1562 (2005)). In all of these applications, it is important to understand the coverage, or loading, of the oligonucleotide (e.g., number of molecules and/or density and/or distribution) on the nanoparticle, and, in many cases, it is favorable to have higher DNA loadings.

Higher DNA loading has the potential to dramatically impact biodetection and nanotherapeutics. The first biodetection assays using DNA functionalized gold nanoparticles involved colorimetric readout strategies (Elghanian, et al. Science, 227:1078-1081 (1997); Storhoff, et al. J. Am. Chem. Soc., 120:1959-1964 (1998); and Reynolds, et al. J. Am. Chem. Soc. 122:3795-3796 (2000)). These procedures were inspired by an aggregation induced red-to-blue color transition attributed to a dampening and red shifting of the nanoparticle surface plasmon resonance (SPR) band. Since the development of the initial colorimetric assays, DNA functionalized gold (Au) nanoparticles have become a central component in a wide variety of schemes that use readout strategies including fluorescence, radioactivity, quartz crystal microbalance, Raman spectroscopy, light scattering, and electrical signal (Zhao, et al. J. Am. Chem. Soc., 125:11474-11475 (2003); Xu, et al. Nucleic Acids Res., 33:e83 (2005); Weizmann, et al. Analyst, 126:1502-1504 (2001); Patolsky, et al. J. Am. Chem. Soc., 122:418-419 (2000); Cao, et al. Science, 297:1536-1539 (2002); Cao, et al. J. Am. Chem. Soc., 125:14676-14677 (2003); Taton, et al. Science, 289:1757-1760 (2000); and Park, et al. Science, 295:1503 (2002)).

In addition, the bio-bar-code method is a strategy that has made a marked impact on the field of gold nanoparticle-based biodiagnostics by providing a protocol to detect proteins, DNA, and other biomolecules at remarkably low concentrations both serially and in a multiplexed format (Nam, et al. Science, 301:1884-1886 (2003); Stoeva, et al. J. Am. Chem. Soc. 128:8378-8379 (2006); Nam, et al. J. Am. Chem. Soc., 126:5932-5933 (2004); Stoeva, et al. Angew. Chem. Int. Ed., 45:3303-3306 (2006); Georganopoulou, et al. P Natl Acad Sci USA, 102:2273-2276 (2005); and Thaxton, et al. Anal. Chem., 77:8174-8178 (2005)). The high sensitivity of this assay stems from an indirect amplification of the target sequence by the sizable number of DNA strands that can be loaded on a single gold nanoparticle. Therefore, the amount of DNA on each nanoparticle directly correlates to the amount of amplification possible and the sensitivity attainable in this system.

Moreover, recent work in the area of nanotherapeutics has demonstrated the use of DNA functionalized gold nanoparticles as antisense agents for intracellular gene regulation. The nanoparticles act as a non-toxic and highly efficient antisense agent that by virtue of cooperative binding properties can very effectively scavenge mRNA within the cell (Lytton-Jean, et al. J. Am. Chem. Soc., 127:12754-12755 (2005) and Rosi et al., Science, 312:1027-1030 (2006)). In addition, tight packing of the DNA on the surface of the nanoparticle likely plays a role in the inhibition of its degradation by nucleases. This opens the door for the use of functionalized gold nanoparticles in several very efficient gene regulation therapies.

Thus, the diagnostic and therapeutic applications of oligonucleotide-modified nanoparticles benefit from the ability to maximize and tailor the amount of DNA on the gold nanoparticle surface.

### SUMMARY

Disclosed herein is a method of preparing nanoparticles having oligonucleotides on the nanoparticle surface. More specifically, the method disclosed herein produces a higher density of oligonucleotide on the nanoparticle surface than prior known methods. The method comprises admixing the nanoparticle and an oligonucleotide having a spacer portion and a recognition portion, under conditions sufficient to form a covalent bond between the nanoparticle surface and the oligonucleotide. The spacer portion includes a moiety that is suitable for covalent attachment to the nanoparticle. Typically, that moiety is a thiol. The conditions sufficient to form the covalent bond can comprise use of a phosphate buffer and a “fast” aging process, wherein the salt, a metal chloride (typically sodium chloride), is increased in concentration to at least about 0.5 M over a time period of up to about 24 hours. This method provides a nanoparticle having oligonucleotides on its surface, wherein the oligonucleotides have a density of at least about 14 pmol/cm², at least about 20 pmol/cm², at least about 30 pmol/cm², at least about 40 pmol/cm², or at least about 50 pmol/cm².

### BRIEF DESCRIPTION OF THE FIGURES

FIG. 1 shows the effect of different concentrations of sodium chloride (NaCl) on DNA loading per particle for DNA having a polyethylene glycol (PEG) spacer, a thymine spacer (T₁₀), or an adenosine spacer (A₁₀).

FIG. 2 shows the effect of sonication on DNA loading per particle for DNA having a PEG spacer, a T₁₀ spacer, or an A₁₀ spacer, on 15 nm, 30 nm, 50 nm, 80 nm, 150 nm, and 250 nm gold nanoparticles.

FIG. 3 shows DNA loading as a function of nanoparticle size at 1.0 M NaCl for DNA containing an A₁₀, T₁₀, or PEG spacer, where the dashed line shows the theoretical values of DNA loading assuming a density fixed to that of a 15 nm nanoparticle.

FIG. 4 shows the effect of fast salting and slow salting conditions on the amount of DNA loading per particle over a range of NaCl concentrations.

FIG. 5 shows the effect of the presence of sodium dodecylsulfate (SDS) on the amount of DNA loading per particle over a range of NaCl concentrations.

FIG. 6 shows the effect of various surfactants (SDS, Tween 20, and Carbowax) on the amount of DNA loading per 15 nm gold nanoparticle over a range of NaCl concentrations, wherein the DNA has a A₁₀ spacer and wherein sonication is used.

FIG. 7 shows the effect of varying the cation of the salt (potassium, lithium, and sodium chloride) on DNA loading per 13 nm gold nanoparticle, wherein the DNA has a T₁₀ spacer.

FIG. 8 shows the effect of phosphate buffer compared to Tris buffer on DNA loading per 13 nm gold nanoparticle over a range of NaCl concentrations, wherein the DNA has a T₁₀ spacer.

FIG. 9 shows the effect of 0 minutes, 1.5 minutes, and 10 minutes of sonication for 30 nm and 50 nm particles on DNA loading per particle.

FIG. 10 shows a liquid chromatography trace of the DNA subjected to sonication, indicating that sonication does not degrade the DNA.

FIG. 11 shows the melt curve for free DNA having a T₁₀ spacer and its complement, which was sonicated prior to hybridization, where Tₘ of the transition is 49° C. and its full width at half maximum (FWHM) is 9° C. The inset shows the first derivative of the melting curve.

FIG. 12 shows the melt curve for free DNA having a T₁₀ spacer and its complement, which was not sonicated prior to hybridization, where Tₘ of the transition is 49° C. and its full width at half maximum (FWHM) is 8° C. The inset shows the first derivative of the melting curve.

FIG. 13 shows the effect of sonication on the stability of the gold-thiol bond, wherein 15 nm gold nanoparticles loaded with DNA were sonicated for various periods of time, after the removal of excess DNA.

FIG. 14 shows a transmission electron microscopy (TEM) image of 80 nm Au nanoparticles after salting, sonication, and washing steps.

FIG. 15 shows the effect of sonciation and heating on DNA loading at a range of NaCl concentrations for 13 nm gold nanoparticles, wherein the DNA has a T₁₀ spacer.

### DETAILED DESCRIPTION

Disclosed herein is full quantification of the loading of oligonucleotides on a range of gold nanoparticle sizes. The dependence of oligonucleotide loading on a nanoparticle is disclosed with respect to: salt concentrations from 0 to 1.0 M sodium chloride and time period for increasing the salt concentration; adenine (A), thymine (T), and non-DNA base (e.g., polyethylene glycol (PEG)) spacers (the region of the oligonucleotide between the recognition sequence and the thiol functionality used to attached the oligonucleotide to the gold nanoparticle); sonication; negatively charged buffer vs. positively charged buffer; and increased temperature. Importantly, the DNA loading obtained from these parameters on several sizes of gold nanoparticles (15, 30, 50, 80, 150 and 250 nm in diameter) can be determined. Through these studies, two parameters have been identified as influencing the amount of DNA loading on a nanoparticle, e.g., the use of PEG as a spacer and the use of sonication. Even large 250 nm particles can be both stably and heavily loaded with DNA. These large nanoparticles have the potential to be loaded with several orders of magnitude more DNA strands than the smaller particles (e.g., 13-30 nm) that often are used in biodiagnostic assays which rely on gold clusters as probes.

The oligonucleotide-modified nanoparticles disclosed herein can have oligonucleotide densities of at least about 14 pmol/cm². An oligonucleotide density is the amount of molecules of oligonucleotide per surface area of the nanoparticle. In certain embodiments, the density of the nanoparticles is at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 pmol/cm². The density can be about 14 to about 20 pmol/cm², about 19 to about 60 pmol/cm², about 20 to about 50 pmol/cm², or about 22 to about 35 pmol/cm². The total number of oligonucleotide molecules on a nanoparticle is dependent upon the nanoparticle size and can be measured analytically, according the to methods disclosed below and as outlined in Scheme 1. By measuring the amount of fluorescence of a fluorescent tag attached to an oligonucleotide, one can determine the total number of molecules of oligonucleotide attached to the surface of a nanoparticle. Also, given the total amount of surface area of larger nanoparticles is higher than for smaller nanoparticles, the total number of oligonucleotides that can be attached to a larger nanoparticle is higher. This is best seen in the theoretical line of FIG. 3. The greater the surface area, the greater the number of oligonucleotide molecules possible to be attached to the nanoparticle.

The method disclosed herein comprises admixing the nanoparticles and an oligonucleotide under conditions sufficient to form a covalent bond between the nanoparticle surface and the oligonucleotide. The oligonucleotide comprises at least two portions: a spacer portion and a recognition portion. The spacer portion of the oligonucleotide is designed such that it can bind to the nanoparticle, which typically is inclusion of a moiety suitable for covalently binding to the nanoparticle. As a result of the binding of the spacer portion of the oligonucleotide to the nanoparticle, the recognition portion of the oligonucleotide is spaced away from the surface of the nanoparticle and is more accessible for hybridization with the recognition portion's target. The length and sequence of the spacer portion providing good spacing of the recognition portion away from the nanoparticle can be determined empirically. It has been found that a spacer portion comprising at least about 7 nucleobases, preferably 10 to 30 nucleobases, gives good results. Also given good results is a spacer portion comprising a polymer, typically PEG, having a molecular weight of about 250 Da to about 1000 Da, or about 250 Da to about 500 Da.

The spacer portion may have any sequence which does not interfere with the ability of the recognition portion to become bound to the nanoparticle or to a nucleic acid or oligonucleotide target. For instance, the spacer portions should not be sequences complementary to each other, to that of the rest of the oligonucleotide, or to that of a target. Preferably, the nucleobases of the spacer portion are all adenines, all thymines, all cytidines, or all guanines, unless this would cause one of the problems just mentioned. More preferably, the bases are all adenines or all thymines.

In some embodiments, the spacer moiety comprises a functionality other than a nucleobase, such as, for example, a polymer which does not interfere or impede the oligonucleotide attachment to the nanoparticle and/or the interaction of the recognition portion of the oligonucleotide with its corresponding target. A non-limiting example of such polymers contemplated for use as a spacer portion as disclosed herein is polyethylene glycol (PEG). In embodiments where PEG is employed as a spacer moiety, the molecular weight of the PEG typically is about 250 Da to about 500 Da, but may be up to about 1000 Da.

The spacer portion typically comprises a thiol (SH) functional group, which is used to attach the oligonucleotide to the nanoparticle surface. However, other functional groups also can be used. Oligonucleotides functionalized with thiols at their 3′-end or 5′-end readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. Chem. Commun. 555-557 (1996) which describes a method of attaching 3′ thiol DNA to flat gold surfaces. The thiol moiety also can be used to attach oligonucleotides to other metal, semiconductor, and magnetic colloids and to the other types of nanoparticles described herein. Other functional groups for attaching oligonucleotides to solid surfaces include phosphorothioate groups (see, for example, U.S. Pat. No. 5,472,881 for the binding of oligonucleotide-phosphorothioates to gold surfaces), substituted alkylsiloxanes (see, for example, Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides having a 5′ thionucleoside or a 3′ thionucleoside may also be used for attaching oligonucleotides to solid surfaces.

The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

Oligonucleotides also may, include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Other nucleobases can be used in the oligonucleotides disclosed herein, including those disclosed in U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, each of which is incorporated herein by reference in its entirety.

The conditions sufficient to form a covalent bond between the spacer portion of the oligonucleotide and the nanoparticle surface are discussed in greater detail below, and can include: a “fast” salting process where the final salt concentration is at least about 0.5 M; use of a surfactant; sonication; use of phosphate as buffer; and combinations thereof.

A “fast salting process” refers to a process of increasing a salt concentration to a final salt concentration in a period of time that is up to about 24 hours, where the salt concentration is increased in short time increments (e.g., every 20-60 minutes). Prior salting methods, termed “slow,” have used greater time periods to achieve the final salt concentration, typically about 48 hours, where the salt concentration was increased every 12 hours. The final salt concentration in the “fast salting process,” as used herein, can be attained in less than about 20 hours, less than about 18 hours, less than about 16 hours, less than about 14 hours, less than about 12 hours, less than about 10 hours, less than about 8 hours, or less than about 6 hours. The final salt concentration in the “fast salting process” typically is at least about 0.5M, but can be at least about 0.55 M, at least about 0.6 M, at least about 0.65 M, at least about 0.7 M, at least about 0.75 M, at least about 0.8 M, at least about 0.85 M, at least about 0.9 M, at least about 0.95, or up to about 1.0 M. The salt used typically is a metal chloride, and preferably sodium chloride. Other metal chlorides also can be used, such as lithium chloride or potassium chloride.

The use of a surfactant when attaching oligonucleotides to the nanoparticle surface is also disclosed herein. Use of a surfactant is preferred when a fast salting process is used, but a surfactant can be used for any of the methods disclosed herein. The surfactant can be used to stabilize the oligonucleotide-functionalized nanoparticles, especially at the higher salt concentrations. Non-limiting examples of surfactants contemplated for use in the disclosed methods include sodium dodecyl sulfate (SDS), polyoxyethylene(20)sorbitan monolaurate (Tween 20), polyethylene glycol (Carbowax), Triton X-100, lithium dodecyl sulfate, potassium dodecyl sulfate, and mixtures thereof.

Gold nanoparticles (15, 30, 50, 80, 150, and 250 nm) were functionalized with fluorophore labeled, alkanethiol modified oligonucleotides (SEQ ID NO: 1—having an adenosine spacer; SEQ ID NO: 2—having a thymine spacer; or SEQ ID NO: 3—having a PEG spacer). Nanoparticle concentration was determined using UV-vis spectroscopy. Nanoparticle-bound oligonucleotides were liberated into solution by addition of dithiothreitol (DTT) and quantified using fluorescence spectroscopy, as depicted in Scheme 1.

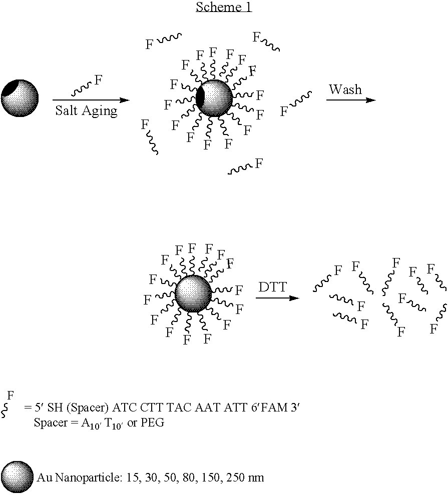

### Effect of Buffer

Since the initial functionalization of gold nanoparticles with modified DNA in 1996, modifications have been made to the procedure. Recent publications have described a “fast” salt aging process where the final salt concentration of the solution is reached within several hours as opposed to 40 hours, as originally described. This “fast” salt aging is made possible by the addition of surfactant molecules prior to salt aging. These molecules decrease the tendency of the nanoparticles to aggregate and coalesce, particularly at high salt concentrations. Surfactants also increase the stability of larger nanoparticles (e.g., those greater than 100 nm) during the salt aging process.

The initial fast salt aging step was to ensure that these modifications did not detrimentally affect the loading of DNA on Au nanoparticles. Control experiments indicated DNA loading was independent of salting rate. The presence of surfactant molecules improves reproducibility and slightly increases the DNA loading (about 39% more strands/particle at 1.0 M NaCl) because the surfactant reduces sticking of the nanoparticles either to each other and to the surface of the glass vials. The type of surfactant used (SDS, Tween 20, or Carbowax) did not affect DNA loading. In addition, no change in loading was observed when varying the salt cation (Li⁺, Na⁺, K⁺), while keeping the CF anion constant.

Experiments also were performed that compared DNA loading in a phosphate buffer to that in a Tris buffer, which contain a positively charged amine group. The DNA loading was found to be slightly lower (about 16% less strands/particle at 1.0 M NaCl) in the Tris buffer than in the phosphate buffer. This result is attributed to electrostatic interactions between the positively charged Tris molecules and the negatively charged DNA backbone (Mazur, et al. J. Phys. Chem. B, 105:1100-1108 (2001)). Because of this interaction, each DNA strand occupies a larger effective volume at the surface of the nanoparticle. This added bulk decreases the amount of DNA on the particle surface because fewer DNA strands can pack into a given space. Thus, a phosphate buffer provides a higher density of oligonucleotide on a gold nanoparticle than a Tris buffer.

### Effect of Salt Concentration

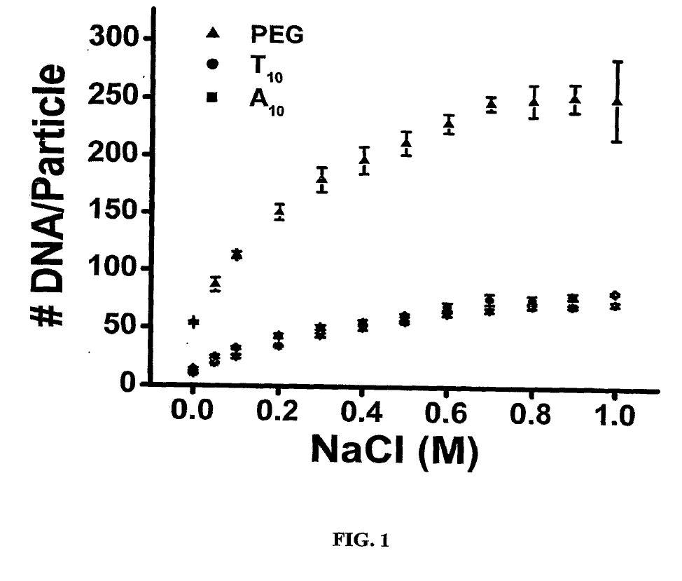

Initial loading experiments were performed to investigate DNA loading as a function of NaCl concentration (0 to 1.0 M), (FIG. 1; 15 nm nanoparticles). While higher salt concentrations previously were reported to result in higher DNA loading, this study was extended to determine the salt concentration at which maximum loading is achieved. The number of DNA strands was found to increase with the addition of salt, and to plateau between 0.7 and 1.0 M NaCl. The initial increase is attributed to a reduction of repulsive forces between the DNA strands as the concentration of counter ions is increased. At high salt concentrations (about 0.7 M), maximum screening is achieved between neighboring DNA strands and the loading remains relatively constant. These results indicate that gold nanoparticle probes should be salt aged to at least 0.7 M NaCl to obtain maximum loading.

### Effect of Spacer Composition

The effect of spacers on DNA loading was also investigated. Typically, probe DNA sequences are designed with a spacer region between the alkanethiol and the recognition sequence. The spacer serves the purpose of moving the recognition sequence further from the particle surface, thereby reducing steric crowding of this region during hybridization steps. The loading of DNA sequences containing a 10-adenine or thymine oligonucleotide (A₁₀, T₁₀), or PEG spacer having roughly the same length as 10 DNA bases was measured as a function of NaCl concentration between 0 and 1.0 M, (FIG. 1). Interestingly, a dramatic increase in loading was observed with DNA containing the PEG spacer (about 250 DNA strands/particle) compared to the A₁₀ and T₁₀ spacers (about 70 and about 80 DNA strands/particle, respectively). The data pertaining to DNA loading with the nucleobase spacers agrees with previous studies which found DNA containing a poly-T spacer, e.g., T₁₀, to give higher loadings compared to DNA with a poly-A spacer, e.g., A₁₀ (Demers, et al. Anal. Chem., 72:5535-5541 (2000)). Similar effects from salt aging and spacer type were observed for all other nanoparticle sizes.

Not wishing to be bound by theory, it is postulated that the influence of the spacer type on DNA loading is due to the interactions between neighboring spacer regions and the spacer region with the gold surface. Spacer regions composed of nucleotide bases can exhibit interstrand repulsion due to the negatively charged phosphate/sugar backbone, thus restricting DNA loading. In addition, the tendency of DNA bases to interact with gold can cause the DNA to partially lie on the gold surface, further reducing DNA loading. The adenine nucleobase has a much stronger relative affinity for gold than the thymine nucleobase (Demers, et al. J. Am. Chem. Soc., 124:11248-11249 (2002); Kimura-Suda, et al. J. Am. Chem. Soc., 125:9014-9015 (2003); Parak, et al. Nano. Lett., 3:33-36 (2003); Kryachko, et al. J. Phys. Chem. B, 109:22746-22757 (2005); Oestblom, et al. J. Phys. Chem. B, 109:15150-15160 (2005); Petrovyhk, et al. J. Am. Chem. Soc., 128:2-3 (2006); and Storhoff, et al. Langmuir, 18:6666-6670 (2002)). As a result, DNA strands containing the A₁₀ spacer are more likely to lie on the Au nanoparticle surface compared to DNA containing the T₁₀ spacer.

The spacer region containing the PEG chain behaves differently. For example, molecules with thiolated-PEG moieties have been shown to form self-assembled monolayers (SAMs) on gold surfaces (Pale-Grosdemange, et al. J. Am. Chem. Soc., 113:12-20 (1991)). Additionally, the PEG spacer is less bulky that an A₁₀ or a T10 spacer, which can allow for a greater number of oligonucleotides to be attached to the surface of a nanoparticle. The lack of intermolecular repulsions between neighboring PEG moieties, the decreased interactions between the PEG and the gold surface, and the decreased bulk of the PEG spacer can all contribute to the higher packing densities for a PEG-spacer oligonucleotide (Levin, et al. Anal. Chem., 78:3277-3281 (2006) and Latham, et al. Langmuir, 22:4319-4326 (2006)). These factors translate to substantially higher DNA loading (about 3 times more) on 15 nm gold nanoparticles for strands containing a PEG spacer compared to the nucleobase spacers (FIG. 1).

### Effect of Sonication

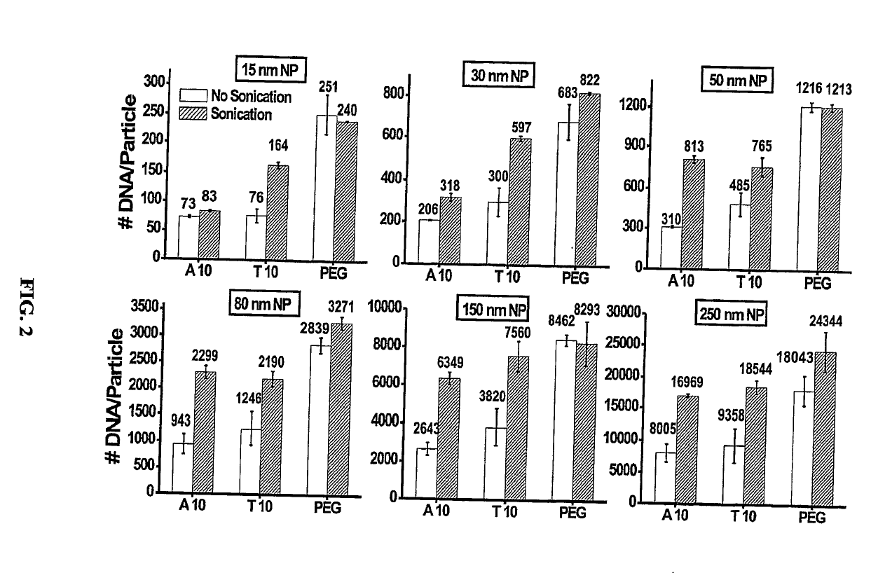

DNA loading was further increased when the gold nanoparticles were sonicated during the salt aging process (FIG. 2). On average, loading was found to roughly double for the A₁₀ and T₁₀ nucleobase spacers. This increase in loading was slightly less for the A₁₀ spacer on the smaller particles (15 and 30 nm). Very little effect was seen on the DNA loading using the PEG spacer. Similar effects of sonication were observed for all nanoparticles sizes. Sonication can disrupt the interactions between the DNA bases and the gold surface, and thereby create room for additional thiolated DNA to attach to the exposed gold nanoparticle surface, and increase DNA loading. This effect was not observed with DNA containing the PEG spacer due to the reduced affinity of PEG for gold. Control experiments revealed that increasing the duration of sonication does not substantially increase loading. In addition, nanoparticles salt aged at elevated temperatures (55° C. for 10 min after each salt addition) showed increased loading, with results comparable to those obtained with sonication.

Control experiments determined that the DNA strands were not altered by this level and duration of sonication. HPLC analysis determined that DNA strands were not chemically degraded after sonication. In addition, identical melting temperatures (Tₘ) were found when comparing the melting transition of sonicated and unsonicated DNA. Control experiments also determined that additional sonication, after the removal of excess DNA, did not cause nanoparticle-bound DNA to detach. Transmission electron microscopy (TEM) showed that the nanoparticles remain highly monodisperse after sonication and washing steps.

### Nanoparticle Size

The relationship between nanoparticle size and DNA loading was also explored. Large gold particles (e.g., 150 and 250 nm in diameter) were successfully stabilized with alkanethiol modified DNA. As the nanoparticle diameter increases from 15 to 250 nm, the DNA loading increases by two orders of magnitude for all spacers (FIG. 3). For example, in the case of the PEG spacer, the DNA loading on a single 15 nm nanoparticle is about 250 strands, while that on a 250 nm nanoparticle is about 25,000 strands. However, the DNA density is largest for the 15 nm particles and decreases as the particle size increases, Table 1, below. The dashed lines in FIG. 3 represent the theoretical loading for each spacer assuming a fixed density equal to that of a 15 nm nanoparticle. The divergence from the theoretical loading density becomes more pronounced as particle size increases. This effect may be due to the decrease in the curvature of the nanoparticle surface as the particle size increases. This causes the DNA strands to be closer together and intensifies interstrand repulsion. A decrease in DNA density previously was observed when comparing a high-curvature nanoparticle surface to a flat Au substrate.

TABLE 1

| | A₁₀ Spacer | T₁₀ Spacer | PEG Spacer | Surface |
|---|---|---|---|---|
| | pmol/cm² | pmol/cm² | pmol/cm² | Area (cm²) |
| 15 nm | 19 | 38 | 56 | 7 × 10⁻¹² |
| 30 nm | 19 | 35 | 48 | 28 × 10⁻¹² |
| 50 nm | 17 | 19 | 26 | 78 × 10⁻¹² |
| 80 nm | 19 | 20 | 27 | 201 × 10⁻¹² |
| 150 nm | 15 | 18 | 19 | 706 × 10⁻¹² |
| 250 nm | 14 | 16 | 21 | 1963 × 10⁻¹² |

The deviation of actual loading from theoretical loading is dependent on the type of spacer. For the 250 nm nanoparticles, loading values for the A₁₀, T₁₀, and PEG spacer are 65, 60, and 26% of theoretical loading, respectively. When the curvature of the surface is high (e.g., with smaller particles), the effective footprint of the DNA dictates the maximum density at which the DNA strands can pack. Therefore, DNA containing the PEG spacer can pack very densely. This results in a much higher theoretical loading for the PEG spacer. However, as the curvature decreases (larger particles) the repulsion between the DNA strands plays a more substantial role, and the density is less affected by spacer type.

The larger gold particles, e.g., those having a diameter of at least about 150 nm, behave differently during the salt aging possess compared to the smaller particles. Typically, when about 0.6 M NaCl is reached during the salt aging process, the larger particles precipitate from solution. At this time, the solution turns clear, and small aggregates are observed at the bottom of the vial. These aggregates do not redisperse with heating (up to 80° C.), but will resuspend (returning to original color) after gentle sonication (less than 5 seconds). However, when the particles are resuspended in NANOpure® water or a salt concentration below about 0.3 M NaCl, sonication is not necessary. This reversible aggregation may be attributed to an increase in screening between the particles (higher salt concentration) that allows surface interactions to dominate. However, because the particles are protected by the surface bound DNA, the particles do not coalesce to form gold aggregates, and the aggregation is reversible with sonication. When the 1.0 M NaCl PBS buffer is replaced with NANOpure® water, the screening between the particles decreases and the particles redisperse.

In general, DNA loading can be increased by salt aging at least about 0.7 M NaCl and/or by using a PEG moiety as a spacer chain in place of the more common nucleobase (e.g., A or T) spacers. The loading can be further increased by sonicating the DNA/nanoparticle solution during the salt aging process, particularly when an A or T spacer is used. These methods have allowed for much higher loadings of oligonucleotides on nanoparticles. Furthermore, the larger particles (e.g., 150 nm or greater) can carry a substantially greater amount (about 2 orders of magnitude) of DNA compared to smaller nanoparticles (e.g., 13-30 nm).

### EXAMPLES

Gold nanoparticles were purchased from Ted Pella (Redding, Calif.). Oligonucleotides were purchased from Integrated DNA Technologies, Inc. (Coralville, Iowa) (5′HS-SPACER-ATC CTT TAC AAT ATT 6′FAM 3′, where spacer is derived from A₁₀, T₁₀, or [(CH₂CH₂O)₆-phosphoramidite]), SEQ ID NO: 1, SEQ ID NO: 2, or SEQ IQ NO: 3, respectively. Dithiothreitol (DTT) was purchased from Pierce Biotechnology, Inc. (Rockford, Ill.). NAP-5 columns (Sephadex G-25 DNA grade) were purchased from G. E. Healthcare (Piscatiway, N.J.). Carbowax 20 M was purchased from Supelco, Inc. (Bellefonte, Pa.). All other salts and reagents, unless specified, were purchased from Sigma-Aldrich (St. Louis, Mo.). Clear 96-well plates (Costar 3696) and black shell, clear bottom 96-well plates (Costar 3603) were purchased from Corning, Inc. (Corning, N.Y.). NANOpure® H₂O (>18.0 MΩ), purified using a Barnstead NANOpure® Ultrapure water system, was used for all experiments.

Absorbance measurements of oligonucleotides and gold nanoparticles were collected using a Bio-Tek Synergy HT Microplate Spectrophotometer. Fluorescence measurements were performed on a Molecular Devices Gemini EM Microplate Spectrofluorometer. All sonication was performed using a Branson 2510 sonicator.

### Preparation of Alkanethiol Oligonucleotide-Modified Gold Nanoparticles

Gold nanoparticles were functionalized with fluorophore (fluorescein, 6′FAM) modified alkanethiol oligonucleotides. Prior to use, the disulfide functionality on the oligonucleotides was cleaved by addition of DTT to lyophilized DNA and incubated at room temperature for 1 hour (0.1 M DTT, 0.18 M phosphate buffer (PB), pH 8.0). The cleaved oligonucleotides were purified using a NAP-5 column. Freshly cleaved oligonucleotides were added to gold nanoparticles (1 OD/1 mL) and the concentration of PB and sodium dodecyl sulfate (SDS) were brought to 0.01 M and 0.01% respectively. The oligonucleotide/gold nanoparticle solution was allowed to incubate at room temperature for 20 min. The concentration of NaCl was increased to 0.05 M using 2 M NaCl, 0.01 M PBS while maintaining an SDS concentration of 0.01%. The oligonucleotide/gold nanoparticle solution then was sonicated for approximately 10 seconds (s) followed by a 20 minute (min) incubation period at room temperature (about 20-25° C.). This process was repeated at one more increment of 0.05 M NaCl and for every 0.1 M NaCl increment thereafter until a concentration of 1.0 M NaCl was reached. The salting process was followed by incubation overnight at room temperature. To remove excess oligonucleotides, the gold nanoparticles were centrifuged and the supernatant was removed, leaving a pellet of gold nanoparticles at the bottom. The particles then were resuspended in 0.01% SDS. This washing process was repeated for a total of five supernatant removals.

To determine the number of oligonucleotides loaded on each particle, the concentration of nanoparticles and the concentration of fluorescent DNA in each sample were measured. The concentration of gold nanoparticles in each aliquot was determined by performing UV-visible spectroscopy measurements. These absorbance values then were related to the nanoparticle concentration via Beer's Law (A=εbc). The wavelength of the absorbance maxima (λ) and extinction coefficients (ε) used for each particle size are as follows: 15 nm, λ=524 nm, ε=2.4×10⁸ L/(mol*cm); 30 nm, λ=526 nm, ε=3.0×10⁹ L/(mol*cm); 50 nm, λ=531 nm, ε=1.5×10¹⁰ L/(mol*cm); 80 nm, λ=545 nm, ε=6.85×10¹⁰ L/(mol*cm); 150 nm, λ=622 nm, ε=2.19×10¹¹ L/(mol*cm); 250 nm, λ=600 nm, ε=5.07×10¹¹ L/(mol*cm).

In order to determine the concentration of fluorescent oligonucleotides in each aliquot, the DNA was chemically displaced from the nanoparticle surface using DTT. The displacement was achieved by adding equal volumes of oligonucleotide-functionalized gold nanoparticles and 1.0 M DTT in 0.18 M PB, pH 8.0. The oligonucleotides were released into solution during an overnight incubation and the gold precipitate was removed by centrifugation. To determine oligonucleotide concentration, 100 μL of supernatant was placed in a 96-well plate and the fluorescence was compared to a standard curve. Because the 6′FAM fluorophore is sensitive to pH, the oligonucleotide samples for the standard curve were prepared with the same 1.0 M DTT buffer solution. During the fluorescence measurement, the fluorophore was excited at 495 nm and the emission was collected from 530 to 560 nm.

The number of oligonucleotides per particle for each aliquot was calculated by dividing the concentration of fluorescent oligonucleotides by the concentration of nanoparticles. All experiments were repeated three times using fresh samples to obtain reliable error bars.

### Salting Experiments

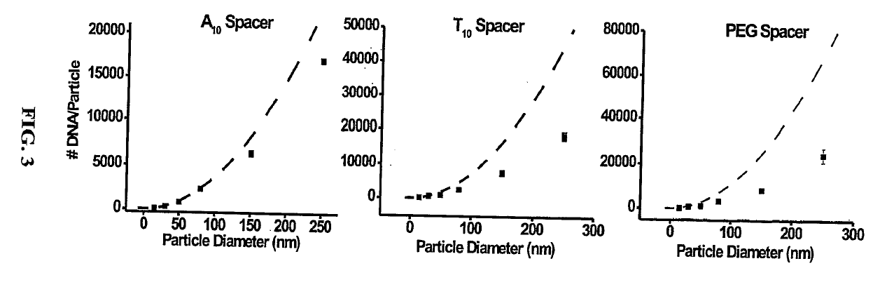

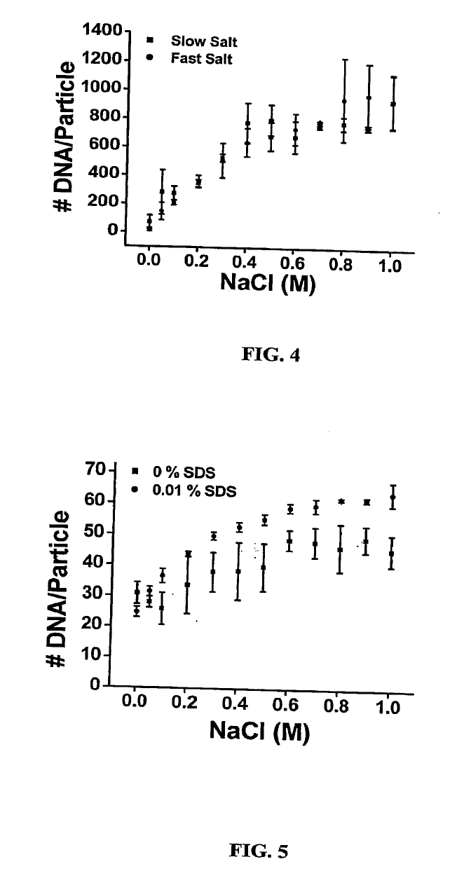

“Slow” salting, wherein the salt concentration is increased every 12 hours, and “fast” salting, wherein the salt concentration is increased every 20 minutes, each were performed on a sample of 80 nm gold nanoparticles using DNA with an A₁₀ spacer. FIG. 4 shows that loading of the DNA on the nanoparticles is the same within experimental error, irrespective of the salt loading.

### Surfactant Experiments

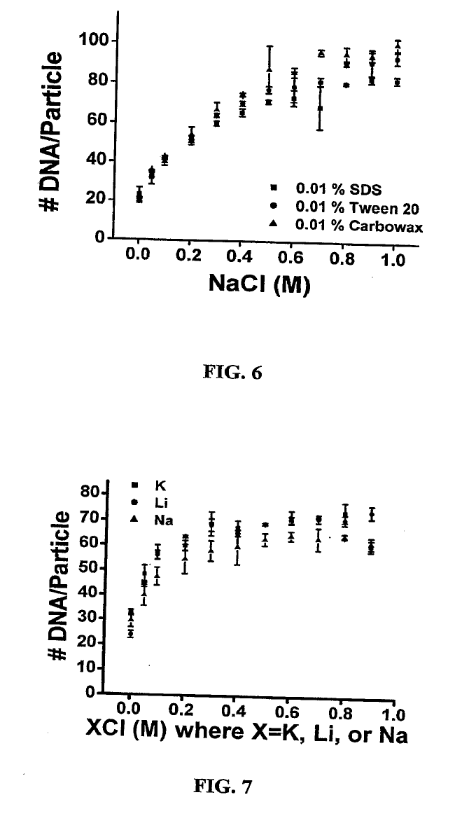

The loading of DNA also was found to be slightly higher in the presence (FIG. 5) of surfactant but was independent of the type of surfactant used (FIG. 6). In FIG. 5, aliquots of gold nanoparticles (13 nm) were loaded with DNA (A₁₀ spacer) through the fast salt aging process both with and without 0.01% SDS. Gold nanoparticles (13 nm) were used because they can be easily stabilized to high salt concentrations without the use of surfactant molecules. The results suggest that the presence of a small quantity of surfactant aids in maximizing DNA loading on the nanoparticle because it prevents the nanoparticles from adhering either to each other or to the walls of the reaction vial. In this way, surfactants allow a more homogeneous DNA coating to be obtained on the nanoparticles.

Three different surfactants (i.e., SDS, Tween-20, Carbowax-20M) were used. All of these surfactants differ significantly in terms of charge, size, and functional groups. Despite these chemical and physical differences, DNA loading on the Au nanoparticles was similar for the surfactants investigated (FIG. 6). When these experiments were performed with Tween-20 and Carbowax-20M, the washing steps were performed with 0.01% solutions of the respective surfactant.

### Cation Experiments

The effect of the salt cation also was investigated. Salts having a corresponding chloride anion (Cl⁻) were chosen to probe the effect of the salt cation on DNA loading. Because the phosphate/sugar backbones of the DNA strands are negatively charged, the positively charged ions interact with the DNA. Sodium, lithium, and potassium were investigated. These cations have the same charge with different ionic radii. The same DNA loading was obtained with all three salts (FIG. 7). In these experiments, all buffer and surfactant solutions were prepared with the corresponding cation. For example, lithium dodecyl sulfate and lithium phosphate were used as surfactant and buffer in the lithium studies.

Divalent cations were also investigated, e.g., Mg²⁺ and Ca²⁺. For these experiments, the loading of the nanoparticles with DNA could not be accomplished using the same protocol. With the first addition of either a small amount of MgCl₂ or CaCl₂, the gold nanoparticles irreversibly aggregated. In the case of Mg²⁺, which has a higher charge density than Ca²⁺, the aggregates were smaller in size, very compact, and dark purple in color. In the case of Ca²⁺, the aggregates were fluffy, less compact, and red in color. These fluffy aggregates can be temporarily disassembled with sonication or vortexing, but quickly re-aggregate after the physical stimulus was removed. It is possible that these doubly charged ions allow for linking between multiple DNA strands, which would lead to the observed aggregation.

### Buffer Effects

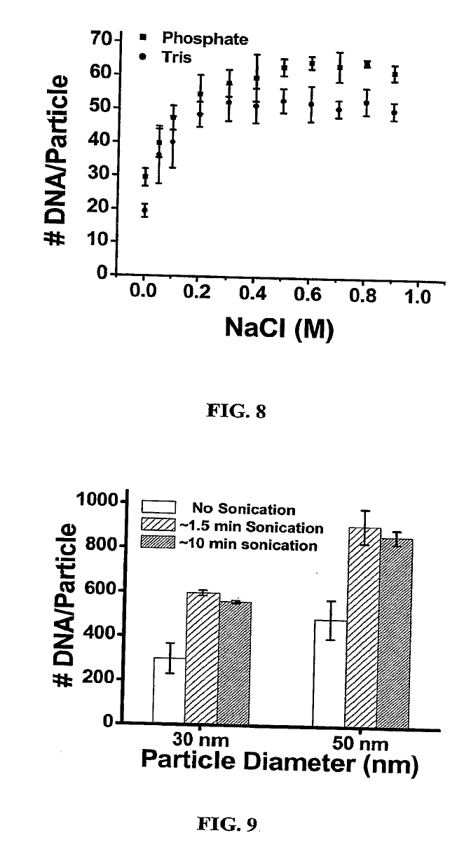

The effects of buffer molecules on the DNA loading of nanoparticles also were elucidated. Several different buffers can be used depending on the desired pH range necessary for a given experiment. The buffer molecules have widely different structures and are chosen such that they do not react with either the gold nanoparticle surface or the DNA in solution. FIG. 8 shows that a Tris buffer has slightly lower loading than a phosphate buffer. In these experiments, Tris dodecyl sulfate (Tris-DS) was used as the surfactant molecule.

### Sonication

Several control experiments were performed to ascertain the effects of sonication on DNA loading. These experiments were designed to test whether extended sonication time (longer than 10 seconds after each salt addition) would result in a additional increases in DNA loading on nanoparticles. At the completion of salt aging (at 1.0 M NaCl), sonication of the nanoparticles was performed for an additional 10 minutes before removal of excess DNA. Increased sonication did not increase DNA loading (FIG. 9). This implies that brief sonication (e.g., sonication of several seconds) provides sufficient energy to disrupt the DNA bases on the surface of the nanoparticle to the fullest extent that loading is affected.

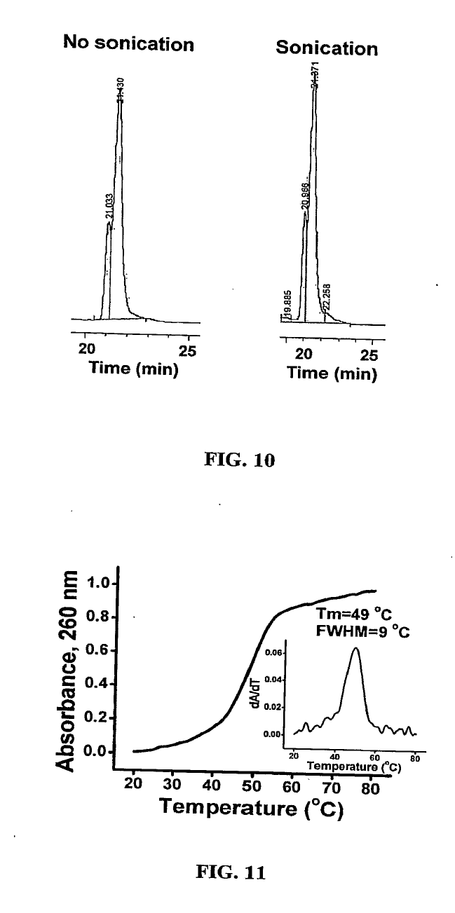

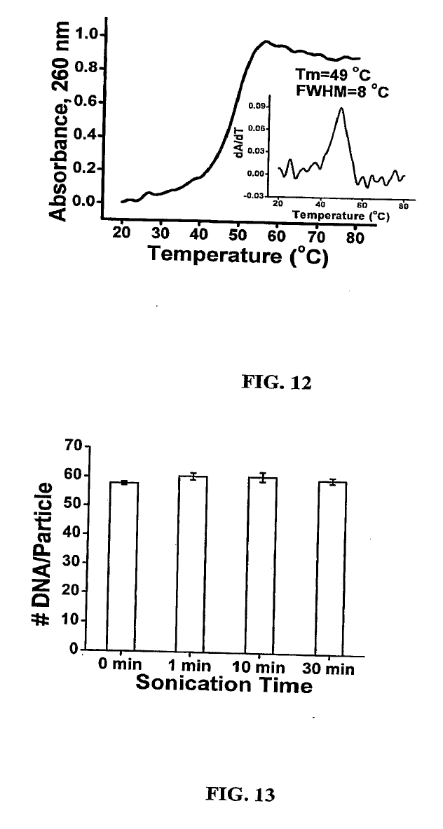

To determine whether sonication damages the DNA stands, analysis of the DNA using HPLC and melting experiments were performed on the DNA both before and after sonication. Free DNA (not attached to a nanoparticle) was sonicated for the same duration and at the same intensity that was used to load the nanoparticles. HPLC showed that the DNA was not chemically degraded (FIG. 10). In addition, when hybridized to its complementary strand overnight, the melting temperature (Tₘ) at 260 nm remained the same (FIG. 11 and FIG. 12). These experiments indicate that little or no DNA damage occurs during sonication.

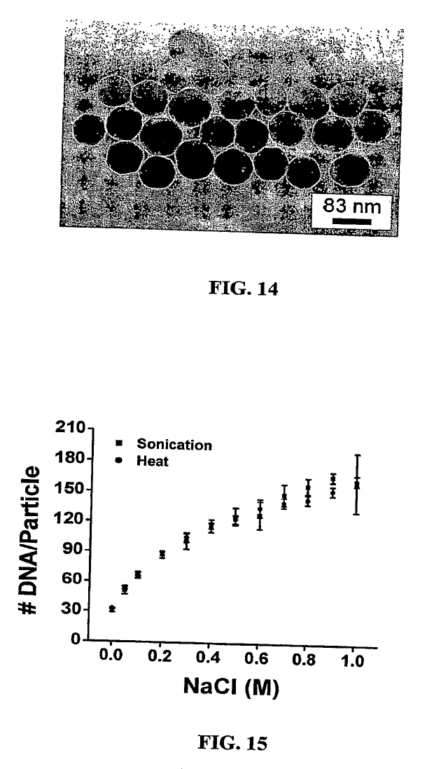

Also investigated was the possibility that sonication disrupts the Au-thiol bond, which would cause DNA to be released from the surface of the nanoparticle. Nanoparticles were salt aged and excess DNA was removed. Aliquots were collected, then sonicated for an additional 1, 10, or 30 minutes and cleaned again. DNA loading of the nanoparticles after extended sonication was similar to normal sonication (FIG. 13). However, if sonication did disrupt the Au-thiol bond in the loading protocol described herein, excess DNA strands would be free to immediately replace any displaced DNA. The nanoparticles were analyzed to assess whether sonication and high salt concentrations damaged their surfaces. For all nanoparticle sizes investigated, TEM images revealed no nanoparticle damage occurred. FIG. 14 shows a representative TEM of 80 nm Au nanoparticles.

### Temperature Effects

The effects due to heating during DNA loading were also investigated. A brief heating step (55° C. for 10 minutes) was performed after each increase in salt, rather than a brief sonication step. DNA loading was the same in each scenarios (FIG. 15). Either a temperature increase or sonication, therefore, is effective in increasing the amount of DNA loaded onto a gold nanoparticle surface. Thus, the oligonucleotide can be loaded to the surface of the nanoparticle under heating of about 50° C. to about 70° C. or about 55° C. to about 60° C.

What is claimed:

1. A method of preparing a nanoparticle having oligonucleotides attached thereto comprising
admixing (i) a nanoparticle and (ii) an oligonucleotide having a spacer portion and a recognition portion, under conditions sufficient to form a covalent bond between the nanoparticle surface and the oligonucleotide,
wherein the conditions sufficient to form the covalent bond comprise (a) use of a phosphate buffer, (b) an increasing salt concentration to at least 0.5 M over a time period of up to about 24 hours, and (c) use of a surfactant, and
wherein the oligonucleotide density on the nanoparticle surface is greater than the oligonucleotide density on the nanoparticle prepared in the absence of the conditions (a), (b), and (c).

2. The method of claim 1, wherein the salt concentration is aged to at least about 0.7 M and the time is up to about 12 hours.

3. The method of claim 1, wherein the spacer comprises at least 7 sequential adenosine nucleobases.

4. The method of claim 3, wherein the oligonucleotide density is at least about 14 pmol/cm².

5. The method of claim 3, wherein, when the nanoparticle has a diameter up to about 80 nm, the oligonucleotide density is at least about 17 pmol/cm².

6. The method of claim 1, wherein the spacer comprises at least 7 sequential thymine nucleobases.

7. The method of claim 6, wherein the oligonucleotide density is at least about 15 pmol/cm².

8. The method of claim 6, wherein, when the nanoparticle has a diameter up to about 80 nm, the oligonucleotide density is at least about 19 pmol/cm².

9. The method of claim 1, wherein the spacer comprises polyethylene glycol (PEG).

10. The method of claim 9, wherein the PEG has a molecular weight of about 250 to about 1000 Da.

11. The method of claim 9, wherein the oligonucleotide density is at least about 19 pmol/cm².

12. The method of claim 9, wherein, when the nanoparticle has a diameter up to about 80 nm, the oligonucleotide density is at least about 26 pmol/cm².

13. The method of claim 1, wherein the conditions sufficient to form the covalent bond further comprises sonicating the admixture, heating the admixture to a temperature of about 50° C. to about 70° C., or both.

14. A nanoparticle having oligonucleotides attached to at least a portion of the nanoparticle surface, wherein
when the nanoparticle has a diameter of greater than 100 nm to about 250 nm, the oligonucleotide density on the nanoparticle surface is at least about 14 pmol/cm²; and
when the nanoparticle has a diameter of up to about 100 nm, the oligonucleotide density on the nanoparticle surface is at least about 20 pmol/cm².

15. The nanoparticle of claim 14, wherein the density is at least about 30 pmol/cm².

16. The nanoparticle of claim 15, wherein the density is at least about 50 pmol/cm².
